(19) United States
(12) Patent Application Publication
(10) Pub. No.: US 2012/0165629 A1
(43) Pub. Date: Jun. 28, 2012

(54) SYSTEMS AND METHODS OF MONITORING A PATIENT THROUGH FREQUENCY-DOMAIN PHOTO MIGRATION SPECTROSCOPY

(76) Inventors: Sean Merritt, Lake Forest, CA (US); Marcelo M. Lamego, Coto De Caza, CA (US); Massi Joe E. Kiani, Laguna Niguel, CA (US)

(21) Appl. No.: 13/249,121

(22) Filed: Sep. 29, 2011

Related U.S. Application Data
(60) Provisional application No. 61/388,545, filed on Sep. 30, 2010.

(51) Int. Cl. A61B 5/1455; A61B 6/00

(52) U.S. Cl. 600/323; 600/479

(57) ABSTRACT

FDPM processing provides an amplitude signal and a phase signal at a modulation frequency to improve measurement fidelity during measurement of one or more blood parameters. In an embodiment, a light source modulates light at a modulation frequency around 200 MHz to produce an amplitude and phase plethysmograph, usable to access clinical test data.

### REFERENCE TO RELATED APPLICATIONS

The present application claims priority benefit under 35 U.S.C. §119(e) from U.S. Provisional Application No. 61/388,545, filed Sep. 30, 2010, entitled “Systems and Methods of Monitoring a Patient Through Frequency-Domain Photo Migration Spectroscopy,” which is incorporated herein by reference.

### FIELD OF THE DISCLOSURE

The present disclosure relates to the field of monitoring patients through analysis of absorption data. More specifically, the disclosure relates to frequency-domain photo migration spectroscopy.

### BACKGROUND OF THE DISCLOSURE

Frequency-domain photo migration (“FDPM”) spectroscopy is often used to determine optical properties of turbid samples, including the determination of absorption and scattering properties of the samples. In general, FDPM usually includes irradiating a sample at an air-medium interface with light whose intensity is modulated at variable frequencies, often in the MHz range. A photo-detector receives the light after passing through some or all of the sample, and then outputs electrical signals responsive to the intensities of the received light. These output intensity signals are usually amplitude attenuated and phase delayed, and are often referred to as the amplitude and phase frequency response of the sample. In certain situations, bulk absorption and scattering optical properties of the sample can be determined from the frequency response.

### SUMMARY OF THE DISCLOSURE

Pulse oximetry is a standard-of-care in many patient monitoring environments including surgical, recovery, and general care wards. It is also used in home monitoring, fitness, spot checking, and many other situations where vital signs and blood parameter information is useful for caregiver and/or patient review. In general, a pulse oximetry system includes a sensor with a light source and light detectors. The sensor positions the source and the detector such that when the source irradiates a measurement site with light, the detector can receive the light after attenuation by tissue at the measurement site. The sensor outputs a signal responsive to the attenuation, which is usually preprocessed to, for example, reduce noise, digitize, and in some cases, reduce the amount of available data in the signal. Once preprocessed, one or more microprocessor, controllers or digital signal processors apply one or more processing methodologies to develop, for example, ratio or other data that can be used as an index to organized clinical or other data to determine output measurement values for, for example, oxygen saturation, pulse rate, plethysmographic information, other blood parameters including for example, carboxyhemoglobin, methemoglobin, total hemoglobin, glucose, an indication of hydration, pH, bilirubin, combinations of the same or the like. The indexing or lookup table that associates ratio values with clinical data is often called a calibration curve.

While the foregoing discussion represents a general overview, an artisan will recognize from the disclosure herein many methodologies and monitor technologies capable of developing measurement output data from signals indicative of absorption of light by body tissue. For example, U.S. Pat. No. 6,157,850, owned by Masimo Corp. of Irvine Calif. (“Masimo”) or U.S. Pat. Pub. No. 2010-0030040, owned by Masimo Laboratories, Inc. of Irvine, Calif., discloses many such processing techniques and systems capable of performing those techniques. Moreover, monitoring instruments commercially available from Masimo employ those and other techniques to monitor patients in many of the foregoing monitoring environments.

While pulse oximetry is a proven technology, developers continually seek processing techniques that have the potential to outperform the foregoing processing in special circumstances or even generally across monitoring environments. The present disclosure provides systems and methods of applying FDPM techniques to determine output measurements that in some circumstances may outperform the general pulse oximetry processing techniques disclosed above, whether those oximetry processing techniques are used alone or in parallel, and whether those techniques are employed always, sometimes, or only in predetermined circumstances. Thus, in some embodiments, the FDPM techniques may be part or all of a separate calculation executing in parallel with other calculations, may be part of a system that selects it as a calculation technique from many other techniques available, may stand alone or be incorporated into other parameter calculation techniques, or the like.

In general, instrument components and temperature can adversely affect the FDPM phase response. Thus, FDPM can require instrument specific calibration. Moreover, instrument components, light source intensities and temperature can also adversely affect the FDPM amplitude response. Usually, expensive stable light sources are used to try to create very stable optical power outputs and/or continuous measurement of optical power output. Moreover, traditional FDPM can measure only bulk optical properties. For application in patient monitoring, bulk response is less useful, while the absorption by, for example, arterial blood is more desired.

The present disclosure seeks to overcome some or all of the foregoing challenges by advantageously applying FDPM to determine robust amplitude and phase photoplethysmographic data usable as indexes to clinical data to determine output measurement values for one or more physiological parameters of a monitored patient. In an embodiment, normalization of an FDPM amplitude signal can reduce that signal's dependency on instrument specific frequency response, temperature, instrument specific light source intensity and/or patient tissue characteristics, such as depth of pigmentation or the like. In an embodiment, normalization of an FDPM phase signal can reduce that signal's dependency on instrument specific frequency response and temperature. After normalization, averaging or other processing techniques can be use to isolate amplitude and phase plethysmographs, which can then be processed with calibration data to determine output measurement values.

For purposes of summarizing the disclosure, certain aspects, advantages and novel features of the disclosure have been described herein. Of course, it is to be understood that not necessarily all such aspects, advantages or features will be embodied in any particular embodiment of the disclosure.

### BRIEF DESCRIPTION OF THE DRAWINGS

A general architecture that implements the various features of the disclosure will now be described with reference to the drawings. The drawings and the associated descriptions are provided to illustrate embodiments of the disclosure and not to limit the scope of the disclosure.

FIG. 1A illustrates a traditional FDPM system usable to determine various optical properties of a turbid sample.

FIG. 1B illustrates an exemplary Bode plot of the amplitude frequency response of the sample of FIG. 1A.

FIG. 1C illustrates an exemplary Bode plot of the phase frequency response of the sample of FIG. 1A.

FIG. 2 illustrates an exemplary block diagram of a monitoring system according to an embodiment of the present disclosure.

FIG. 3 illustrates an exemplary data flow diagram of data processed by one or more digital signal processors of the monitoring system of FIG. 2, according to an embodiment of the present disclosure.

FIG. 4 illustrates an exemplary Bode plot of the amplitude frequency response of the instrument of FIG. 2, according to an embodiment of the present disclosure.

FIG. 5 illustrates an exemplary Bode plot of the phase frequency response of the instrument of FIG. 2, according to an embodiment of the present disclosure.

FIG. 6 illustrates comparative output data from the processing of the system of FIG. 2 versus output data of a traditional pulse oximeter.

FIG. 7 illustrates traditional pulse oximetry processing of plethysmograph data to determine measurement data compared to processing of the system of FIG. 2 to determine potentially more accurate measurement data.

### DETAILED DESCRIPTION OF THE PREFERRED EMBODIMENT

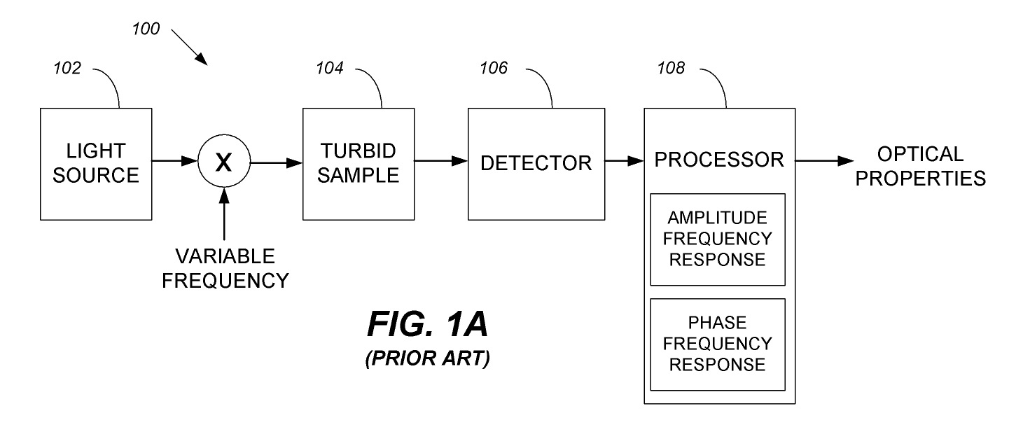

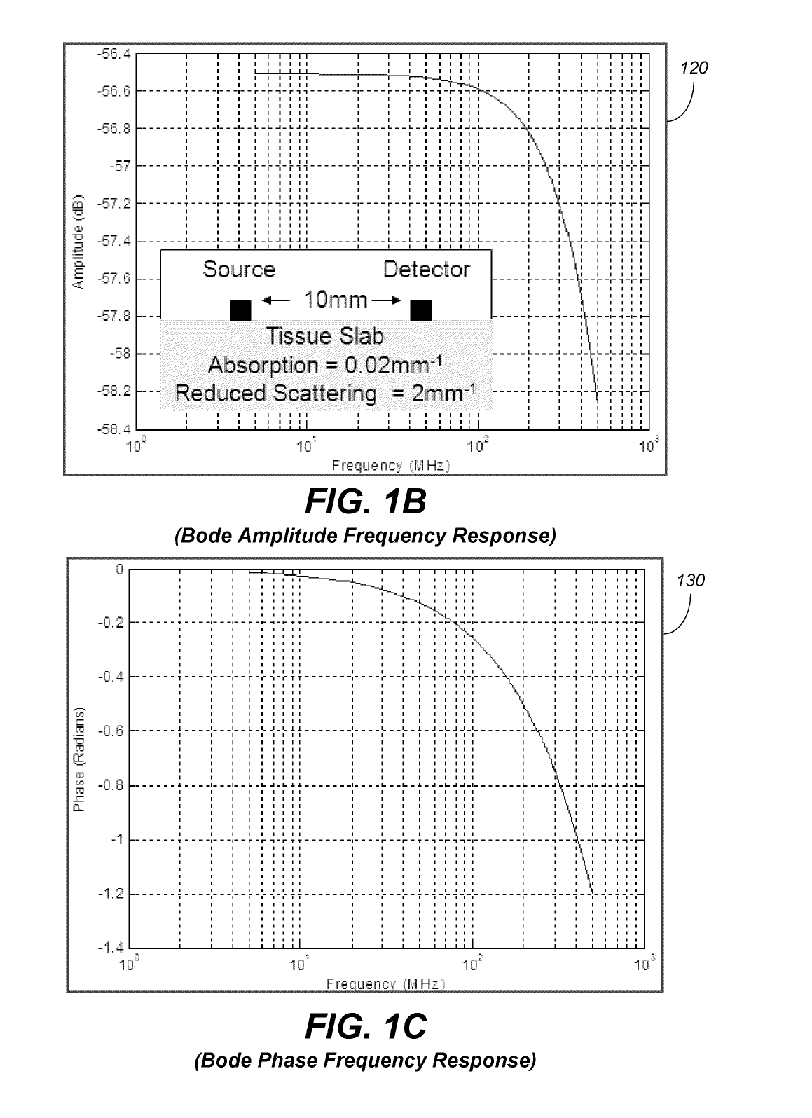

FIG. 1A illustrates a traditional FDPM system 100 including a for example, sinusoidal light source 102 modulated at variable frequencies irradiating a sample 104. After attenuation and scattering of radiation, a detector 106 receives the light and outputs a signal indicative of the attenuation and scattering to a processor 108. The processor 108 processes the signal to determine bulk optical properties, such as, for example, absorption and scattering of the sample. In an embodiment, the processor 108 may advantageously use an amplitude and phase frequency response in its determination.

FIG. 1B illustrates an exemplary Bode plot 120 of the bulk amplitude frequency response of the sample 104 of FIG. 1A. In general, the amplitude plot 120 is a graph of the logarithm of the transfer function of the substantially linear, time-invariant sample versus varied frequency, plotted with a log-frequency axis, to show the system's frequency response. In particular, FIG. 1B shows the amplitude in dB along the y axis and the log-frequency in MHz along the x axis. It is noteworthy that the amplitude plot 120 shows significantly decreasing amplitude attenuation around about 200 MHz.

FIG. 1C illustrates an exemplary Bode plot 130 of the bulk phase frequency response of the sample 104 of FIG. 1A. FIG. 1C shows the phase in radians along the y axis and the log-frequency in MHz along the x axis. It is noteworthy that the phase plot 130 shows significantly decreasing phase delay around about 200 MHz.

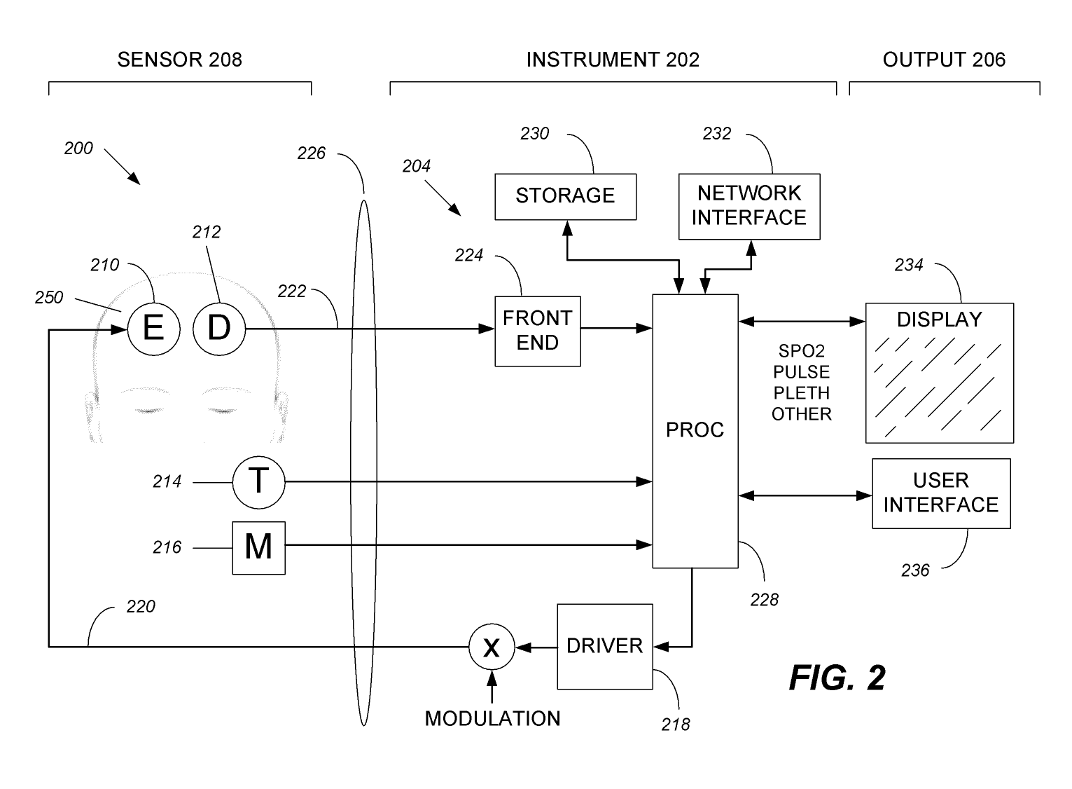

FIG. 2 illustrates an exemplary block diagram of a monitoring system 200 according to an embodiment of the present disclosure. As shown in FIG. 2, the system 200 includes a monitoring instrument 202 including one or more processing boards 204 communicating with a monitor output 206. The processing board(s) 204 communicates with a sensor 208, such as, for example, a noninvasive optical sensor including one or more light sources 210 and one or more light detectors 212. The sensor 208 may also optionally include one or more temperature sensors 214 indicative of light source temperature and/or bulk temperature, and/or include one or more memories 216. The light source 210 may advantageously communicate with one or more drivers 218 whose output 220 may be modulated variably, at desired frequencies, ranges of frequencies, or the like.

While the sensor 208 is shown as a finger sensor positioning the light sources 210 and detector(s) 212 proximate the tissue of a finger, usually such that light shines through the nail bed from the top of the finger through to the bottom, an artisan will recognize from the disclosure herein that the sensor may comprise a wide variety of optical sensors, including for example, a disposable digit, ear or other sensor, a reflectance sensor such as a forehead or other sensor, a partially disposable, partially reusable sensor, or any sensor technology commercially available from Masimo or other well-known oximetry sensor providers.

After irradiation by the light sources 210, the detector 212 outputs a signal 222 responsive to attenuated light from the light sources 210 to a front end 224. In an embodiment, the detector output 222, the emitter or light source driving signal(s) 220 and the optional temperature and memory signals may travel along conductors of a cable 226. An artisan will recognize that some or all of the foregoing signals may be communicated wirelessly or the like.

The front end 224 communicates with one or more digital signal processors, microprocessors, microcontrollers, or the like (hereinafter “processor”) 228. The processor 228 may communicate with the memory 216, the temperature sensor 214, the driver 218, other memory or storage 230, a network interface 232, and the monitor output 206, combinations of the same, or the like. The monitor output 206 may advantageously include one or more displays 234, a user interface 236, or simply format the output for input into external systems.

In general, the processor 228 outputs drive signals to a driver circuit 218, often to control the current applied to the light source 210. The output is combined with a modulation signal comprising a variable frequency, a frequency range, a frequency range above about 100 MHz, a frequency range around 200 MHz, or the like. The output modulated drive signal drives the light source 210, such as, for example, a plurality of same or different LEDs producing light at the same or different wavelengths. In a preferred embodiment, the light source 210 is time division multiplexed such that a single wavelength of light (or OFF) is emitted at any one point in time. The light source may also or alternatively comprise side emitting LEDs, super luminescent LEDs, or the like. As shown in FIG. 2, the sensor 208 may comprise a sensor to be applied to, for example, the index finger of a patient. In other embodiments, the instrument 202 may seek to monitor brain cooximetry or depth of anesthesia or consciousness. The instrument 202 may also or alternatively seek to monitor oximetry measurements for one or more blood analytes or other parameters mentioned above.

In other embodiments recognizable to an artisan from the disclosure herein, the sensor 208 may comprises a transmittance sensor applied to a digit, an ear or ear concha, a septum, the forehead, or the like. In any event, the sensor 210 positions the emitter with respect to the detector 212 where the detector 212 is irradiated by light after attenuation and scattering by body tissue, such as, for example, the illustrated forehead 250.

The detector 212 outputs a signal responsive to the light received, which is communicated to the front end 224. The front end 224 preprocess the signal and communicates the same to the processor 228 that determines, for example, output measurements for the desired physiological parameters of the measurement site.

Although disclosed with reference to the foregoing elements, an artisan will recognize from the disclosure herein other circuits, systems, or processing boards capable of processing sensor output data to display or forward measurement results.

To determine the amplitude response at a given modulated frequency, it is noteworthy that the response is a function of the light source intensity, the instrument attenuation at the modulated frequency, the bulk tissue attenuation at the measurement site, and the pulsating arterial blood attenuation at the modulated frequency. Normalization can remove or at least greatly reduce the effects of differences in source intensity across differing sensors. Operation of the instrument 202 without tissue can provide the frequency response of the instrument 202. After band-pass filtering, the signal represents a normalized plethysmograph at the modulated frequency, which is non-zero and thus, will include phase information. The foregoing normalized plethysmograph at the modulated frequency has been shown to be sensitive to absorption and have better signal quality than traditional pulse oximetry processing by itself. However, with the addition of the phase information, which is sensitive to scattering, the combination of information advantageously reduces errors in determined measurement values.

To determine the phase response at a given modulated frequency, it is noteworthy that the response is a function of the instrument phase shift at the modulated frequency, the bulk tissue phase shift at the measurement site, and the pulsating arterial blood phase shift at the modulated frequency. Normalization can remove or at least greatly reduce the effects of differences in the response across differing instruments. After band-pass filtering, the signal represents a normalized phase plethysmograph at the modulated frequency. The foregoing normalized plethysmograph at the modulated frequency has been shown to be sensitive to scattering.

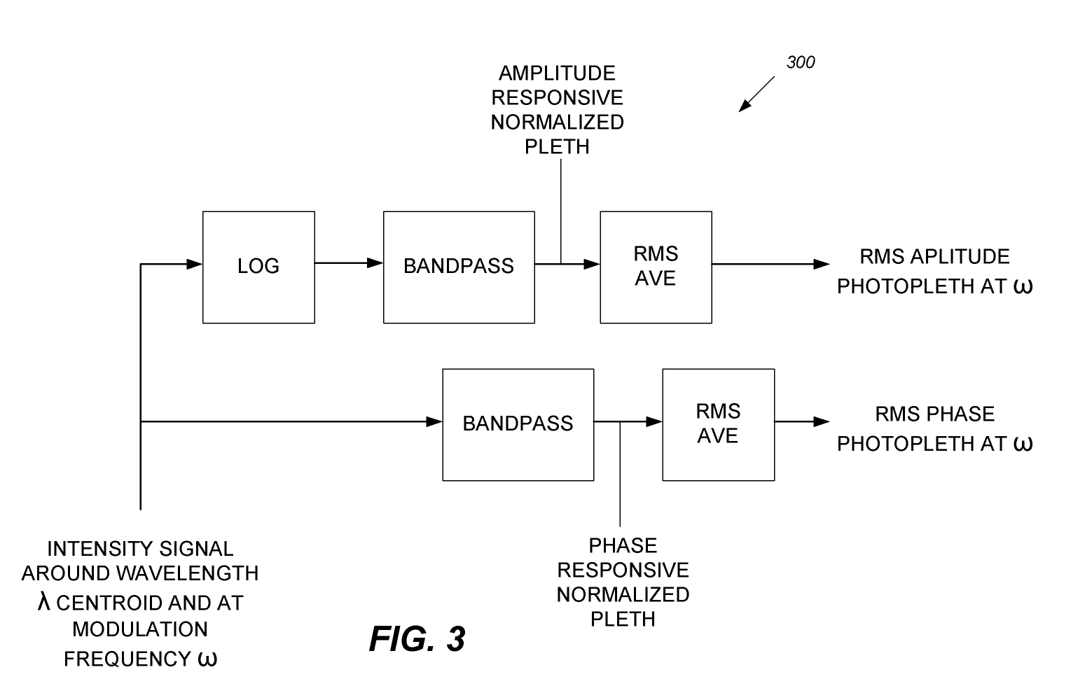

For example, FIG. 3 illustrates an exemplary data flow diagram 300 of data processed by the processor 228 of the monitoring system 200 of FIG. 2, according to an embodiment of the present disclosure. As shown in FIG. 3, the received intensity signal from the detector 212 or the front end 224 is modulated at a given frequency around a given expected emission centroid or wavelength. Taking the log and band-pass filtering the intensity signal provides a normalized plethysmograph responsive to the amplitude response of the bulk tissue at the given modulation frequency, while band-pass filtering the intensity signal also provides a normalize plethysmograph responsive to the phase response at the given modulation frequency. RMS averaging provides a RMS amplitude plethysmograph at the non-zero modulated frequency and a RMS phase plethysmograph at the non-zero modulated frequency.

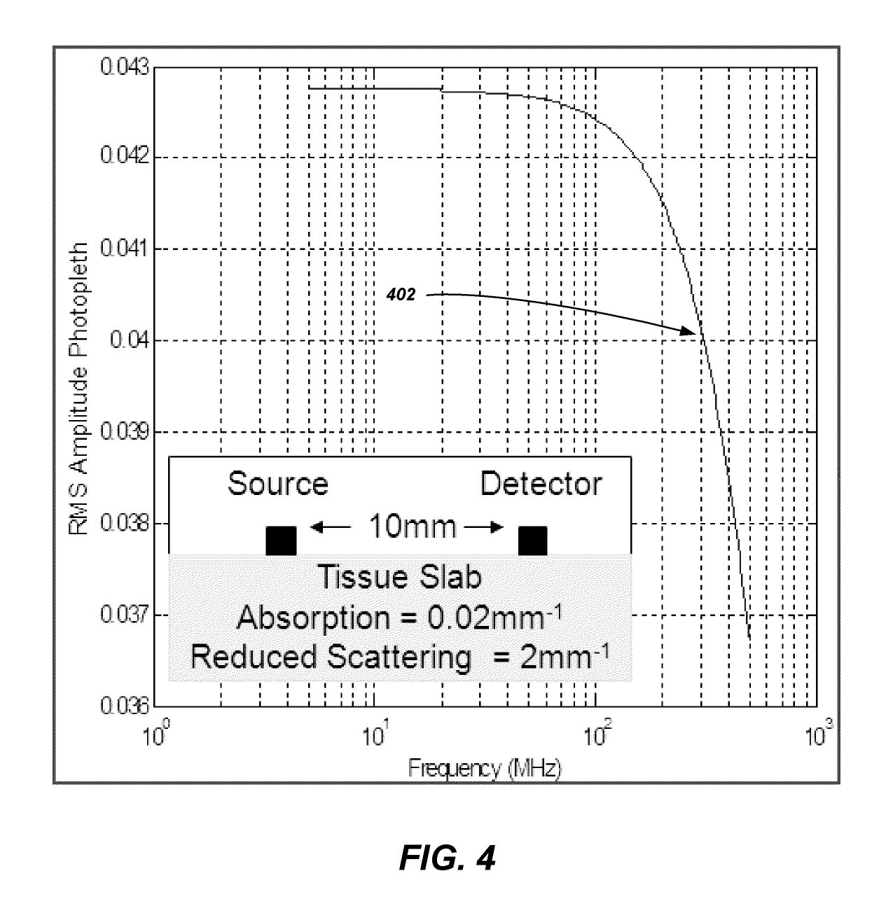

FIG. 4 illustrates an exemplary Bode plot of the amplitude frequency response of the instrument of FIG. 2, according to an embodiment of the present disclosure. It is noteworthy that the variable frequency modulation input creates a relatively narrow amplitude response between about 0.043 dB and about 0.036 dB, indicating a need for more stringent SNR management than conventional pulse oximetry. Various methodologies and component selections known to an artisan from the disclosure herein can be implemented to obtain desired SNR ranges. As shown in FIG. 4, the RMS amplitude photoplethysmograph attenuates dramatically starting around 100 MHz.

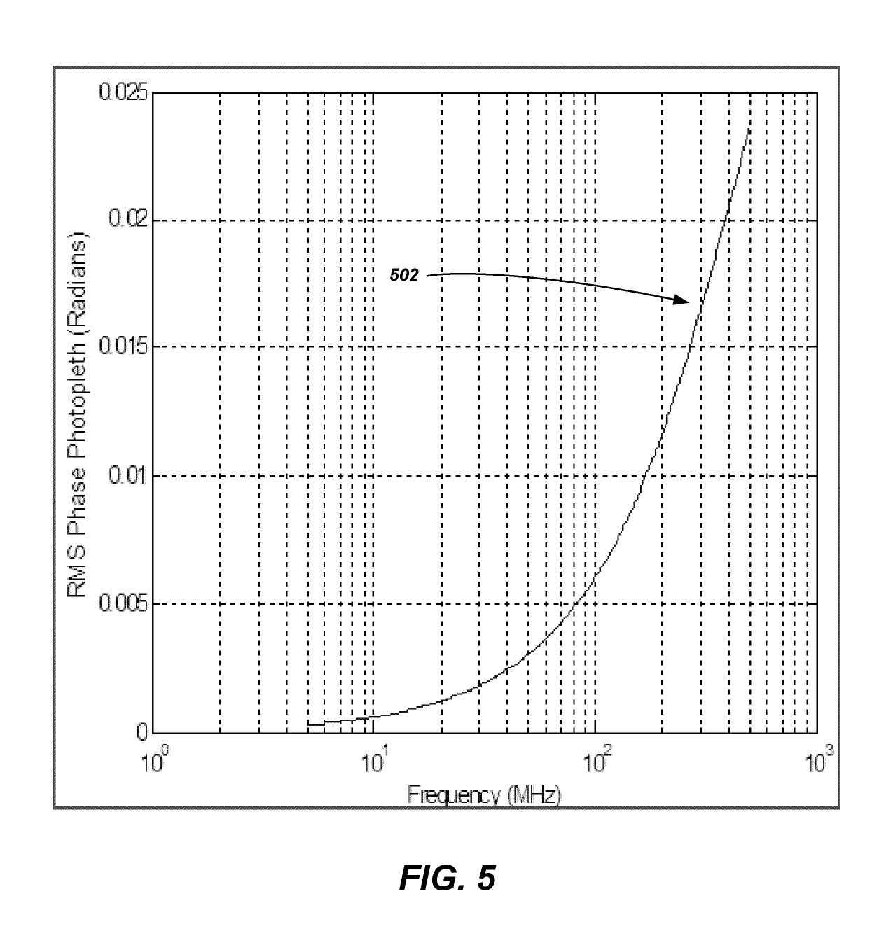

FIG. 5 illustrates an exemplary Bode plot of the phase frequency response of the instrument of FIG. 2, according to an embodiment of the present disclosure. As shown in FIG. 5, the RMS phase photoplethysmograph increases dramatically starting around 100 MHz. Thus, combining the information about frequency response from FIGS. 4 and 5, the modulating frequency of choice should provide robust amplitude response and robust phase response. Thus, as shown in FIGS. 4 and 5, a range of frequencies along the x axis of the amplitude plot provide an amplitude response balanced with a phase response along that same x axis of the phase plot. For example, at around 200 MHz, the amplitude plot of FIG. 4 has an output amplitude response 402 that is roughly as significant as the output phase response 502. Thus, in a preferred embodiment, the modulating frequency is above about 100 MHz. In another embodiment, the modulating frequency ranges from about 100 MHz-about 300 MHz. In another embodiment, the modulating frequency is about 200 MHz.

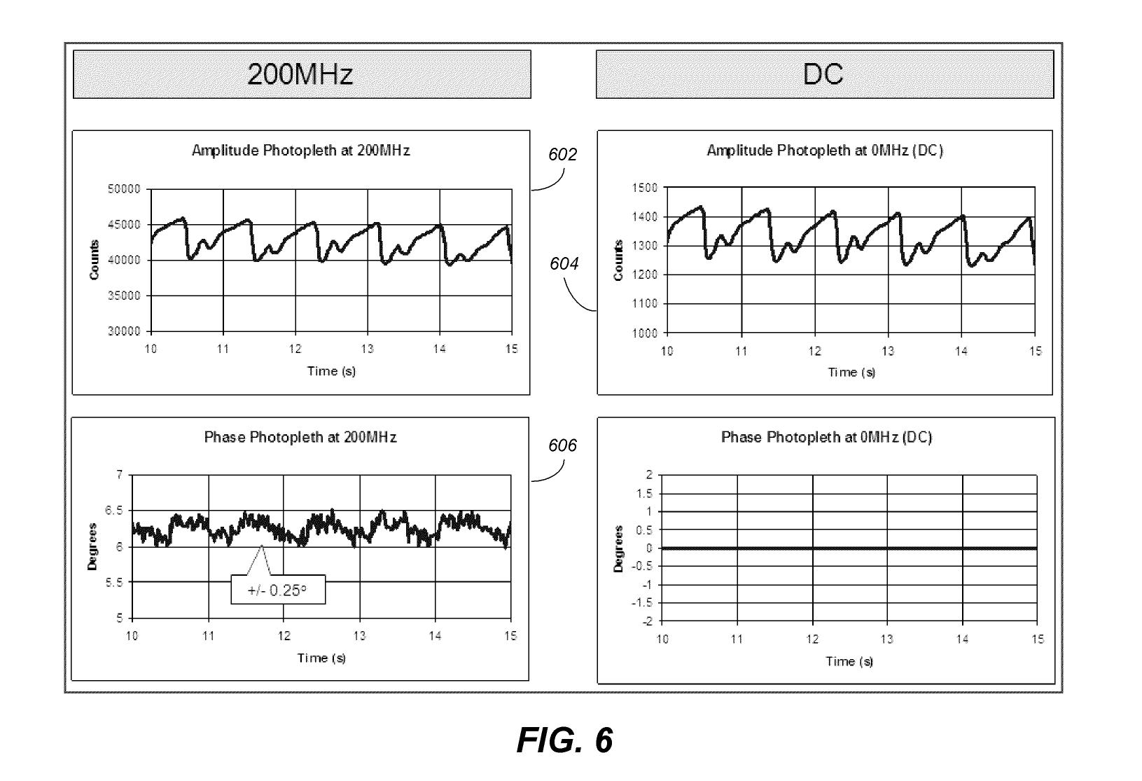

FIG. 6 illustrates comparative output data from the processing of the system of FIG. 2 versus output data of a traditional pulse oximeter. As shown in FIG. 6, the output RMS amplitude plethysmograph 602 at the modulated frequency is substantially similar to the output plethysmograph 604 generally associated with traditional pulse oximetry, that is, with a modulation of zero. However, as also shown in FIG. 6, with FDPM processing, the system 200 also has the output RMS phase plethysmograph 606 providing substantially more information to a processor that can be used in parameter determination.

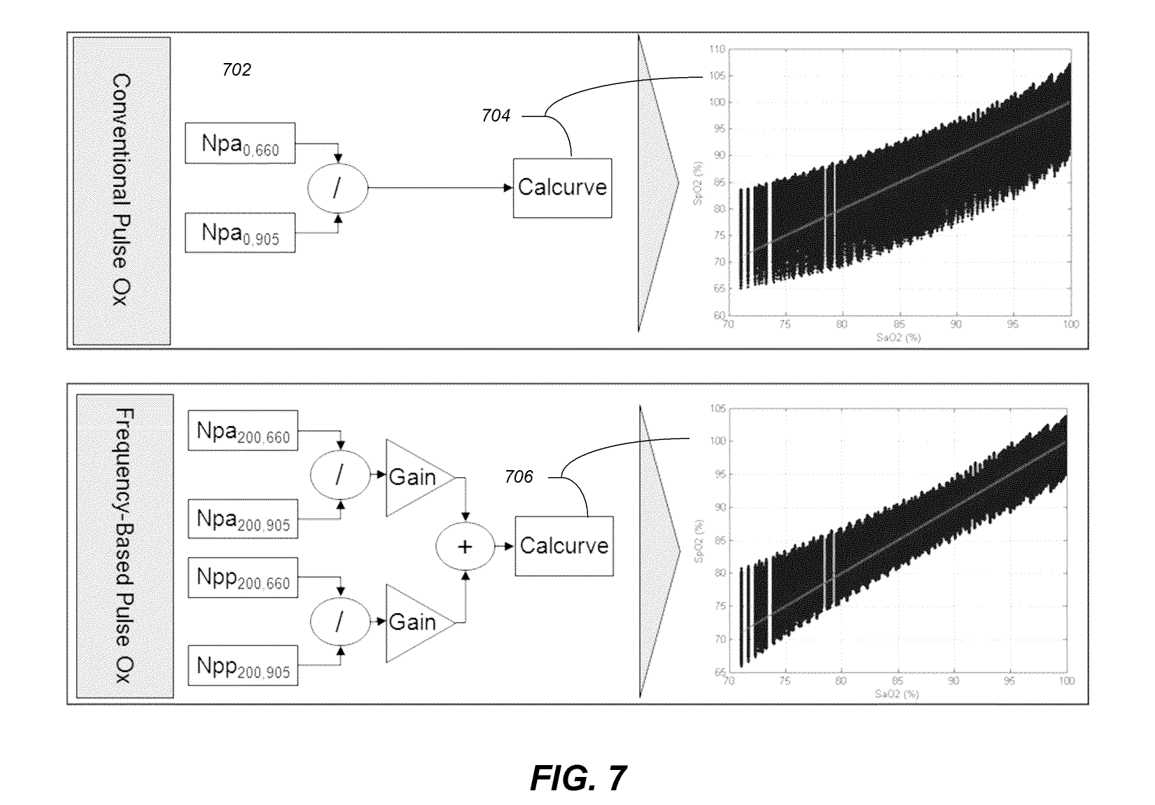

FIG. 7 illustrates traditional pulse oximetry processing of plethysmograph data to determine measurement data compared to processing of the system of FIG. 2 to determine potentially more accurate measurement data. For example, the plethysmograph processed from emitted light at about 660 nm through traditional pulse oximetry 702 is often divided by the plethysmograph processed from emitted light at about 905 nm to create ratio data. The ratio data is used as an index or lookup into clinical data to determine output measurement values. As shown in FIG. 7, the calibration curve 704 from traditional pulse oximetry is fairly wide, corresponding to a larger potential error in measurement values. Meanwhile, as shown in FIG. 7, use of the phase information reduces the error in the calibration curve 706, often substantially.

Although the FDPM system 200 is disclosed with reference to its preferred embodiment, the disclosure is not intended to be limited thereby. Rather, a skilled artisan will recognize from the disclosure herein a wide number of alternatives. Accordingly, the present disclosure is not intended to be limited by the reaction of the preferred embodiments, but is to be defined by reference to the appended claims.

Additionally, all publications, patents, and patent applications mentioned in this specification are herein incorporated by reference to the same extent as if each individual publication, patent, or patent application was specifically and individually indicated to be incorporated by reference.

What is claimed is:

1. A system for determining an output measurement of a physiological parameter of a monitored patient, the system comprising:
a sensor including a light source modulated at a modulation frequency above about 100 MHz and a detector outputting a signal responsive to light from said source after attenuation by body tissue;
a processor receiving a signal responsive to said detector output signal, processing said signal to determine plethysmograph amplitude data and non-zero plethysmograph phase data, and combining said amplitude and phase data to determine an output measurement of said physiological parameter; and
outputting said measurement.

2. The system of claim 1, wherein said processing includes normalizing and band-pass filtering said amplitude data.

3. The system of claim 1, wherein said processing includes band-pass filtering said phase data.

4. The system of claim 1, wherein said processing includes averaging each of said amplitude and phase data.

5. The system of claim 4, wherein said processing includes outputting for display or forwarding to other monitoring devices photoplethysmographs of said amplitude and phase data.

6. A signal processor of a patient monitor, said processor configured to drive a light source at a modulated frequency, to receive a signal indicative of absorption and scattering of body tissue, to process said signal to have an amplitude component and a phase component; combining said amplitude component and said phase component to improve a measurement value.

7. The processor of claim 6, wherein said modulated frequency comprises between about 100 MHz and about 300 MHz.

8. The processor of claim 6, wherein said modulated frequency comprises around about 200 MHz.

9. A method of determining oxygen saturation of a monitored patient, the method comprising:
modulating a light source at a modulation frequency between about 100 MHz and 300 MHz;
receiving a signal from a light detector configured to detected light from said source attenuated by at least pulsing blood of said monitored patient; and
processing said signal with a signal processor of an instrument to substantially reduce a dependency of an amplitude response of said signal on an intensity of said source or on a frequency response of said instrument and to substantially reduce a dependency of a phase of said signal on said frequency response of said instrument, said processing additionally includes determining output measurements for said oxygen saturation based on at least said amplitude and said frequency response and outputting said measurements.

10. The method of claim 9, wherein said modulation frequency comprises about 200 MHz.

11. The method of claim 9, wherein said processing includes normalizing and band-pass filtering said amplitude response.

12. The method of claim 9, wherein said processing includes band-pass filtering said phase response.

13. The method of claim 9, wherein said processing includes averaging each of said amplitude and phase response.

14. The method of claim 9, wherein said outputting said measurements includes displaying photoplethysmographs of said amplitude and phase response.

15. A method of determining confidence in a signal from a noninvasive optical sensor, the method comprising:
modulating a light source at a modulation frequency;
receiving a signal from a light detector configured to detected light from said source attenuated by at least pulsing blood of said monitored patient; and
processing said signal with a signal processor to determine phase information related to said signal, to determine amplitude information related to said signal, and to determine a confidence in said amplitude information based on said phase information, said processing additionally including determining output measurements based on at least said amplitude information and said confidence.

16. The method of claim 15, wherein said modulation frequency comprises about 200 MHz.

17. The method of claim 15, wherein said processing includes normalizing and band-pass filtering said amplitude information.

18. The method of claim 15, wherein said processing includes band-pass filtering said phase information.

19. The method of claim 15, wherein said processing includes averaging each of said amplitude and phase information.

20. The method of claim 15, wherein said processing includes displaying photoplethysmographs of said amplitude and phase information.
